(19) United States
(12) Patent Application Publication
(10) Pub. No.: US 2026/0027032 A1
(43) Pub. Date: Jan. 29, 2026

(54) HAIR IMPROVER AND HAIR IMPROVING COMPOSITION

(71) Applicant: REFINE HOLDINGS CO., LTD., Gifu (JP)

(72) Inventors: Makoto TSUBOI, Tokyo (JP); Masataka OKUBO, Tokyo (JP); Shogo HAMAMOTO, Tokyo (JP)

(73) Assignee: REFINE HOLDINGS CO., LTD., Gifu (JP)

(21) Appl. No.: 19/122,449

(22) PCT Filed: Oct. 26, 2023
(86) PCT No.: PCT/JP2023/038631

§ 371 Date: Apr. 18, 2025

(30) Foreign Application Priority Data
Oct. 28, 2022 (JP) 2022-173101

(51) Int. Cl. A61K 8/73; A61K 8/34; A61Q 5/00; A61Q 5/12

(52) U.S. Cl. CPC A61K 8/73; A61K 8/345; A61Q 5/002; A61Q 5/12

(57) ABSTRACT

Provided is a hair improver that can impart remarkably satisfactory hair moisture content, combability, and hair luster even with the use of an extremely small amount. This hair improver is characterized by containing a sulfated polysaccharide salt having a sulfate group at the 6-position of at least one sugar unit structure and salted with a monovalent cation. This sulfated polysaccharide may have, for example, any one of the following structural formulae:

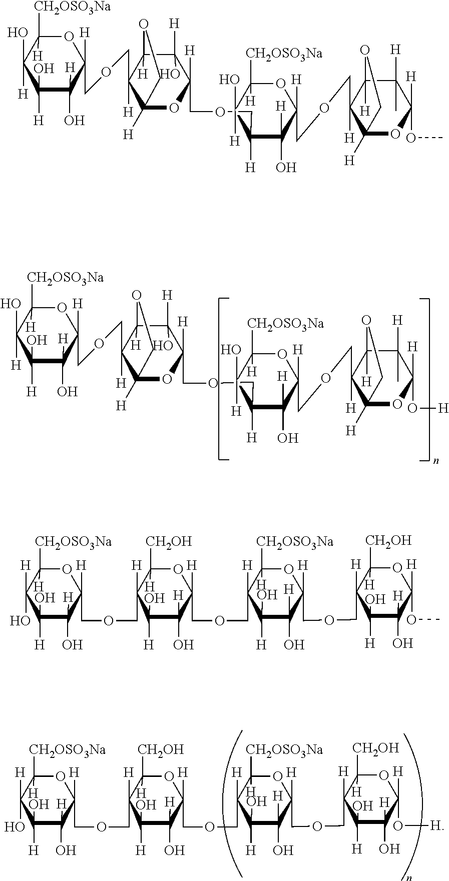

### TECHNICAL FIELD

The present invention relates to a hair improver and a hair improving composition.

### BACKGROUND ART

Since ancient times, hair has been one of the expressions of beauty. In the Heian era, the longer the hair, the more it was a symbol of beauty. Then and now, hair care is important to maintain beautiful hair.

Today, in daily life, in addition to shampooing and brushing, a wide variety of hair care products are used. In addition, people enjoy fashion by styling their hair with coloring, perms, and hair irons, and hair has more opportunities to be damaged.

Shampoo, which used to be used to remove dirt from hair, is now used more frequently, and there is an increasing need to wash hair while caring for it. Hair consists of three parts: the cuticle, medulla, and cortex. The cuticle is on the surface of the hair, the cortex is in the middle, and the medulla is in the center. The cuticle is a transparent, hard, keratinized plate-like cell made of non-keratin protein with a thickness of about 0.5 μm, and has a scale-like layered structure. When the cuticles are aligned, the reflected light on the hair surface is neatly aligned, shining white and beautiful. On the other hand, when hair is damaged, the cuticles peel or come off, the hair surface becomes uneven, and light causes irregular reflection and scattering, resulting in a loss of overall shine.

Causes of hair damage include environmental factors such as ultraviolet rays, friction from brushing, heat treatment from dryers and hair irons, and chemical treatments such as perms and coloring. Since hair has no self-repairing power, daily damage accumulates, causing problems such as dryness, split ends, loss of shine, and difficulty in combing.

Conventionally, silicone and the like have been added to compensate for such damage, but the effect was not sufficient unless the amount added was considerably large. In addition, some silicones are considered to be a part of the environmental burden, and cyclic silicone compounds are regulated by the European Chemicals Regulation (REACH: Registration, Evaluation, Authorization and Restriction of Chemicals) in the EU due to concerns about the ecological environment in aqueous systems, and their use is restricted.

By the way, in Japan, a mixture of funori glue (Japanese glue produced from a glue plant, Gloiopeltis) and wheat flour, which was prepared as a shampoo, was widely used in ancient times. However, when funori glue was used as it is for hair, the effect of beautifying hair was extremely small even if a large amount of funori glue was used.

Furthermore, for example, Patent Literatures 1 to 5 propose hair cosmetics in which sulfated polysaccharides extracted from red algae funori and the like are blended with amino acids, 1-hydroxy-2-pyrrolidone compounds, and amphoteric surfactants. Patent Literature 6 proposes a hair cosmetic containing natural polysaccharides such as funori and betaine. However, sufficient effects could not be obtained with these techniques.

### PRIOR ART DOCUMENTS

### Patent Literatures

Patent Literature 1: JP 57-117139 A

Patent Literature 2: JP 57-183709 A

Patent Literature 3: JP 58-216111 A

Patent Literature 4: JP 59-7109 A

Patent Literature 5: JP 59-7110 A

Patent Literature 6: JP 7-69839 A

### SUMMARY OF THE INVENTION

### Problems to be Solved by the Invention

Therefore, an object of the present invention is to provide a hair improver and a hair improving composition that solves the problems in the prior art as described above. A further object of the present invention is to provide a hair improver and a hair improving composition that can provide very good hair moisture content, combability, and hair shine even with an extremely small amount of use.

### Means for Solving the Problems

As a result of intensive studies to solve the above problems, the present inventors found that there is a polysaccharide molecule with a specific structure that exhibits excellent functionality in the funori polysaccharide obtained by separating funori into molecular units by a specific extraction method, and that it provides very good hair moisture content, combability, and hair shine even with an extremely small amount of use, and completed the present invention.

A hair improver according to a first aspect of the present invention that solves the above problems is characterized by containing a sulfated polysaccharide salt having a sulfate group at the 6-position of at least one sugar unit structure and salted with a monovalent cation.

In the hair improver according to the first aspect of the present invention, the sugar constituent units of the polysaccharide have one or more hexacarbohydrate constituent units selected from the group consisting of fucose, rhamnose, arabinose, galactose, glucose, xylose, mannose, fructose, and ribose, and it is sufficient that at least one hexacarbohydrate unit structure has a sulfate group at the 6-position.

In another preferred embodiment, the sulfated polysaccharide salt has the following compound structural formula:

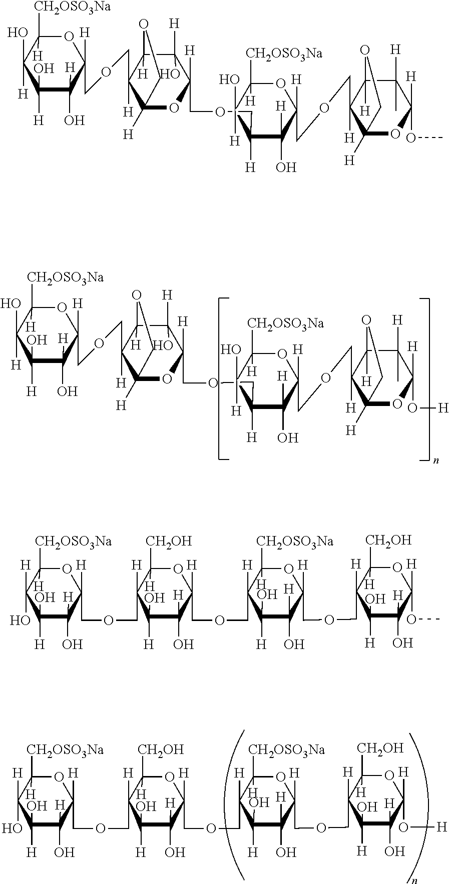

(Provided that, in these formulae, n represents a number from 1 to 20000.)

A hair improving composition according to a second aspect of the present invention that solves the above problems is characterized by containing the hair improver according to the first aspect in an amount of 0.000005% by mass to 0.01% by mass.

In the hair improving composition according to the second aspect of the present invention, an embodiment in which it is an aqueous composition further containing a polyhydric alcohol in an amount of 10 to 50% by mass is shown.

### [Effects of the Invention]

According to the hair improver of the present invention, it is possible to provide very good hair moisture content, combability, and hair shine even with an extremely small amount of use.

### BRIEF DESCRIPTION OF THE DRAWINGS

FIG. 1 is a diagram showing changes in hair moisture content before and after application of the hair improver obtained in one example according to the present invention.

FIG. 2 is a diagram showing changes in hair combability before and after application of the hair improver obtained in one example according to the present invention.

FIG. 3 is a diagram showing changes in the half-value width of hair shine before and after application of the hair improver obtained in one example according to the present invention (A), and a diagram showing changes in hair shine before and after application of the hair improver obtained in one example according to the present invention (B) (photograph as a substitute for a drawing).

FIG. 4 is a diagram showing changes in hair moisture content before and after application of the hair improver obtained in another example according to the present invention.

FIG. 5 is a diagram showing changes in hair combability before and after application of the hair improver obtained in another example according to the present invention.

FIG. 6 is a diagram showing changes in the half-value width of hair shine before and after application of the hair improver obtained in another example according to the present invention (A), and a diagram showing changes in hair shine before and after application of the hair improver obtained in another example according to the present invention (B) (photograph as a substitute for a drawing).

FIG. 7 is a diagram showing changes in hair shine before and after application of the hair improver obtained in still another example according to the present invention (photograph as a substitute for a drawing).

FIG. 8 is a diagram showing changes in hair shine before and after application of a polysaccharide as a comparative example (without sulfate at group the 6-position) (photograph as a substitute for a drawing).

FIG. 9 is a diagram showing changes in hair shine before and after application of a polysaccharide as another comparative example (calcium salt) (photograph as a substitute for a drawing).

FIG. 10 is a diagram showing changes in hair shine before and after application of a general funori extract as another comparative example (photograph as a substitute for a drawing).

FIG. 11 is a diagram showing the analysis results of thin layer chromatography (TLC) of the funori polysaccharide prepared in one example according to the present invention (photograph as a substitute for a drawing).

FIG. 12 is a 13C-NMR spectrum of the funori polysaccharide prepared in one example according to the present invention.

FIG. 13 is an enlarged 13C-NMR spectrum of the main part of the 13C-NMR spectrum of FIG. 12.

### MODE FOR CARRYING OUT THE INVENTION

Next, the present invention will be described in more detail based on embodiments. It should be noted that the embodiments described below do not limit the invention according to the claims, and that not all of the elements and combinations thereof described in each embodiment are essential to the solution means of the present invention.

### Active Ingredient

The hair improver according to the present invention is characterized by containing a sulfated polysaccharide salt having a sulfate group at the 6-position of at least one sugar unit structure and salted with a monovalent cation.

The structure of the polysaccharide constituting the sulfated polysaccharide salt according to the present invention is not particularly limited as long as it has a sulfate group at the 6-position of at least one sugar unit structure, and may be a homopolysaccharide composed of one type of hexacarbohydrate constituent unit, or a heteropolysaccharide containing at least one type of hexacarbohydrate constituent unit and composed of two or more types of sugar constituent units.

In the case of a heteropolysaccharide, the sugar constituent unit other than at least one type of hexacarbohydrate constituent unit may be any of a tricarbohydrate constituent unit, a tetracarbohydrate constituent unit, a pentacarbohydrate constituent unit, or a hexacarbohydrate constituent unit, and two or more sugar constituent units can be constituted by any combination thereof.

The hexacarbohydrate constituent unit contained in the polysaccharide constituting the sulfated polysaccharide salt according to the present invention is not particularly limited, and may be, for example, any of fucose, rhamnose, arabinose, galactose, glucose, xylose, mannose, fructose, and ribose, and those having one or more of these hexacarbohydrate constituent units are included. And it is sufficient that at least a part of the hexacarbohydrate unit structure has a sulfate group at the 6-position.

In the sugar unit structure of the polysaccharide constituting the sulfated polysaccharide salt according to the present invention, as long as at least a part of the hexacarbohydrate unit structure has a sulfate group at the 6-position, the hexacarbohydrate unit structure may have a sulfate group at another position, for example, the 2- or 4-position.

In the sugar unit structure of the polysaccharide constituting the sulfated polysaccharide salt according to the present invention, as long as at least a part of the hexacarbohydrate unit structure has a sulfate group at the 6-position, it is not particularly limited, but the sugar unit structure having a sulfate group at the 6-position is, for example, 0.1 to 100%, preferably 1 to 100%, 10 to 100%, 30 to 100%, 40 to 100%, 50 to 100% of the total sugar unit structure constituting the sulfated polysaccharide salt. It is desirable that it is contained at a ratio such as. Further, if 50% or more, preferably 60% or more, 70% or more, 80% or more, 90% or more, 95% or more of the total sugar unit structure have a sugar unit structure having a sulfate group at the 6-position, there is substantially no difference in effect from that having a sugar unit structure having a sulfate group at the 6-position of 100% of the total sugar unit structure.

The sulfated polysaccharide salt according to the present invention is further salted with a monovalent cation, for example, Na+, K+, NH4+, preferably Na+and K+, particularly preferably Na+. That is, the polysaccharide having a sulfate group as described above does not exhibit a predetermined function due to self-hydrolysis unless it is in the form of a salt, and the counter ion for forming the salt is, for example, a divalent cation such as Ca2+, even if it is a sulfated polysaccharide salt having a sulfate group at the 6-position of the sugar unit structure, the hair improving function of providing hair moisture content, combability, and hair shine is not observed, and it has been clarified that such a hair improving function is exhibited only when it is a salt of a monovalent cation. Therefore, it is preferable that the sulfated polysaccharide salt according to the present invention does not substantially contain divalent cations such as calcium. Here, “substantially not containing” means, for example, that the amount present is 5% or less, 1% or less, 0.1% or less, 0.01% or less, 0.001% or less in molar units, and more preferably an amount that is as close to zero as possible.

Although not particularly limited, examples of the sulfated polysaccharide salt according to the present invention include those having the structural formulae shown below.

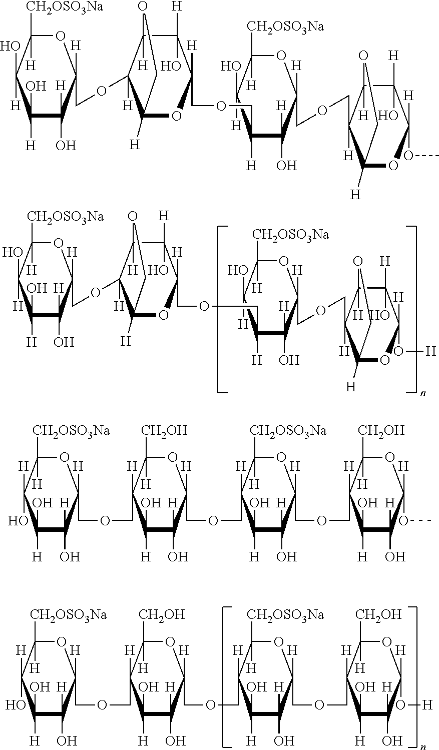

(Provided that, in these formulae, n is 1 to 20000, more preferably, for example, 1 to 10000, 10 to 8000, 100 to 5000, 200 to 4000, 1000 to 2000.)

The sulfated polysaccharide salt according to the present invention may be obtained by synthesis or extracted from various natural raw materials, as long as it has the structure as described above, but it is preferably of natural origin, particularly preferably of funori origin.

### Manufacturing Method

Hereinafter, some examples of the method for producing the sulfated polysaccharide salt according to the present invention will be given, but the method for producing the sulfated polysaccharide salt is not limited to these.

### <Synthesis Method>

Monosaccharides and polysaccharides with 2 or more sugars are acetylated with acetic anhydride/pyridine. The acetylated polysaccharide is produced with ethanol/water, and then lithium hydroxide and tetrahydrofuran (THF) are added to this acetylated polysaccharide and dissolved at 0° C. to room temperature, and sodium hydroxide dissolved in dichloromethane-methanol is added to convert allyluronate and methyluronate to free uronic acid under basic conditions, and at the same time, remove the acetyl group of the hydroxyl group at the 6-position. Subsequently, after reacting with SO₃·NMe₃ at 60° C. overnight, the sulfate group is converted from trimethylamine (Et₃N) salt to Na salt, whereby a 6-position selectively O-sulfated polysaccharide compound can be obtained at a yield of about 80%.

In the case of a polysaccharide, for the 6-position that does not prefer sulfation, benzylation is performed in advance, and the compound O-sulfated at the 6-position is dissolved in ethanol, and catalytically reduced with palladium carbon, whereby a partially 6-position O-sulfated polysaccharide can be obtained. As a result, the sodium salt of the 6-position O-sulfated polysaccharide can be taken out.

In the case of monosaccharides and disaccharides, the sodium salt of the 6-position O-sulfated polysaccharide can be taken out by performing a condensation reaction with a polysaccharide that is not O-sulfated at the 6-position.

The polysaccharide used here is not particularly limited, and examples thereof include hyaluronic acid, cellulose, starch, carrageenan other than lambda (A), alginic acid, fucoidan, and laminaran.

For example, the manufacturing method of the sulfated polysaccharide salt by such synthesis can refer to the descriptions in (1) Noti C, Paz J L D, Polito L, Seeberger P H, Chem. Eur. J., 12, 8664-8686 (2006) and (2) Chen J, Zhou Y, Chen C, Xu W, Yu B, Carbohydr. Res., 343, 2853-2862 (2008). The descriptions of the relevant parts in these documents (1) and (2) are incorporated into this specification by their reference.

<Separation Method from Natural Products 1>

In order separate 6-position to the O-sulfated polysaccharide from plant-based natural raw materials such as algae, the plant natural raw material is powdered, and water is added and dissolved. A salt that becomes a monovalent ion such as sodium chloride is added to the solution and heated. After heating, the salt of the monovalent ion of the 6-position O-sulfated polysaccharide can be taken out by separating by molecular weight.

Here, the plant-based natural raw material means any plant existing in the natural world including algae.

Such plant-based natural raw materials include, but are not limited to, algae such as brown algae (e.g., Laminaria (kelp), Undaria (wakame), Lessonia, Macrocystis (giant kelp), Ecklonia (kajime), Eisenia (arame), Ascophyllum, Durvillaea, etc.), red algae (e.g., Eucheuma, Gloiopeltis, Nemalion, Gelidium (agarophytes), Gracilaria (ogonori), Gloiopeltis (funori), Porphyra (nori), etc.), and green algae (e.g., Codium (mil), Ulva (sea lettuce, aosa), Enteromorpha (aonori), Caulerpa (iwazuta), Monostroma (hitoegusa), etc.). Other plants include, but are not limited to, citrus fruits, sugar beets, and apples. Among these, preferred materials include algae, particularly red algae, and typically, those using funori (Gloiopeltis) as a raw material are exemplified. The salt that becomes a monovalent ion is not particularly limited, and examples thereof include sodium chloride, potassium chloride, sodium sulfate, and potassium sulfate.

Examples of the 6-position O-sulfated polysaccharide derived from plant natural raw materials, for example, seaweed, include λ-carrageenan and funoran.

<Separation Method from Natural Products 2>

As another method for separating the 6-position O-sulfated polysaccharide from plant natural raw materials such as algae, the plant natural raw material is powdered, and water is added and dissolved. An acid such as acetic acid is added to the solution to acidify the solution, and then a desalting operation is performed. An alkali that becomes a monovalent ion such as sodium hydroxide is added to the solution to neutralize it. After neutralization, the salt of the monovalent ion of the 6-position O-sulfated polysaccharide can be taken out by separating by molecular weight.

Here, the plant natural raw materials used are the same as those described above, and the 6-position O-sulfated polysaccharides derived from seaweed are also the same as those described above.

Examples of the acid used include acetic acid, citric acid, malic acid, hydrochloric acid, sulfuric acid, and phosphoric acid, and examples of the alkali that becomes a monovalent ion include sodium hydroxide and potassium hydroxide.

<Separation Method from Natural Products 3>

Other organisms as natural raw materials are not particularly limited, and examples thereof include mammals, horseshoe crabs, squid, and bacteria. Among these, raw materials containing 6-position O-sulfated polysaccharides such as chondroitin sulfate and heparan sulfate as animal intercellular substances are added with water to animal tissues, a buffer solution is added, and then a proteolytic enzyme (protease) is added to decompose proteins. A salt that becomes a monovalent ion such as sodium chloride is added to the tissue lysate that has been treated in this way, and heated. After heating, the salt of the monovalent ion of the 6-position O-sulfated polysaccharide can be taken out by separating by molecular weight.

The salt that becomes a monovalent ion is not particularly limited, and examples thereof include sodium chloride, potassium chloride, sodium sulfate, and potassium sulfate.

Examples of the 6-position O-sulfated polysaccharide derived from animals include chondroitin sulfate, heparan sulfate, keratan sulfate, and heparin.

<Separation Method from Natural Products 4>

As another method for separating the 6-position O-sulfated polysaccharide from raw materials containing the 6-position O-sulfated polysaccharide as an animal intercellular substance, water is added to animal tissues, a buffer solution is added, and then a proteolytic enzyme (protease) is added to decompose proteins. An acid such as acetic acid is added to the tissue lysate that has been treated in this way to acidify the solution, and then a desalting operation is performed. An alkali that becomes a monovalent ion such as sodium hydroxide is added to the solution to neutralize it. After neutralization, the sodium salt of the 6-position O-sulfated polysaccharide can be taken out by separating by molecular weight.

Here, examples of the acid used include acetic acid, citric acid, malic acid, hydrochloric acid, sulfuric acid, and phosphoric acid, and examples of the alkali that becomes a monovalent ion include sodium hydroxide and potassium hydroxide.

Examples of the 6-position O-sulfated polysaccharide derived from animals include chondroitin sulfate, heparan sulfate, keratan sulfate, and heparin, as in the case described above.

### Hair Improving Composition

The hair improver according to the present invention contains the sulfated polysaccharide salt having the specific structure as described above, but in order to make it a hair improving composition containing this as an active ingredient, for example, a composition such as shampoo, rinse, conditioner, hair mist, and hair spray, within a range that does not impair the effects of the present invention, water, oils, surfactants, metal soaps, gelling agents, alcohols, water-soluble polymers, resins, ultraviolet absorbers, ultraviolet protective agents, antibacterial agents, fragrances, pigments, deodorants, salts, pH adjusters, refreshing agents, animal/microorganism-derived extracts, plant extracts, blood circulation promoters, astringents, cell activators, moisturizing agents, enzymes, hormones, vitamins, and other ingredients that are usually used in preparations such as cosmetics, quasi-drugs, and external medicines can be blended. Ingredients listed in ENCYCLOPEDIA OF CONDITIONING RINSE INGREDIENTS (MICELLE PRESS, 1987) and ENCYCLOPEDIA OF SHAMPOO INGREDIENTS (MICELLE PRESS, 1985), and the latest cosmetic science (Yakuji Nippo Co., Ltd., 1988) can be blended. The descriptions of the relevant parts in these documents are incorporated into this specification by their reference.

The hair improver according to the present invention is not particularly limited, but in the hair improving composition as described above, for example, a composition in the form of an aqueous solution, it can exhibit a very good hair improving effect even with a very small amount of 0.000005% by mass or more, preferably 0.00001% by mass or more, based on the total composition. The upper limit of the blending amount is not particularly limited, but from an economical point of view, it is preferably, for example, 0.01% by mass or less, particularly 0.001% by mass, and more preferably 0.0005% by mass or less.

In particular, the hair improving composition according to the present invention is preferably prepared as an aqueous composition for application to hair. Furthermore, in order to make it an aqueous composition, it is desirable to stably retain the sulfated polysaccharide salt having the specific structure as described above as a molecular unit, and to blend polyhydric alcohols for the purpose of preservation.

The water is not particularly limited, and examples thereof include purified water, deionized water, hot spring water, and deep water.

The polyhydric alcohol is not particularly limited as long as it is usually used in external preparations (for example, cosmetics, quasi-drugs, or external medicines). Among polyhydric alcohols, dihydric alcohols or trihydric alcohols are preferable. Specifically, for example, glycol or glycerin is preferable. More specifically, 1,3-butylene glycol, propylene glycol, pentylene glycol (1,2-pentanediol), glycerin, dipropylene glycol, 1,3-propanediol, isoprene glycol (isopentyl diol), etc. are preferably exemplified. Among them, 1,3-butylene glycol, propylene glycol, pentylene glycol, or isoprene glycol is preferable, and 1,3-butylene glycol is particularly preferable. More desirably, it is desirable that it is a naturally derived 1,3-butylene glycol prepared from plant raw materials such as sugar cane. Polyhydric alcohols can be used alone or in combination of two or more.

The lower limit of the content of the polyhydric alcohol is not particularly limited, and may be 1% by mass, 10% by mass, or 30% by mass based on the total mass of the hair improving composition. The upper limit of the content of the polyhydric alcohol is not particularly limited, and may be 99% by mass, 95% by mass, 90% by mass, or 85% by mass based on the total mass of the hair improving composition. As described above, from the viewpoint of stably retaining the sulfated polysaccharide salt having the specific structure as a molecular unit and for the purpose of preservation, it is preferably 8% by mass or more, particularly 10 to 50% by mass, particularly 20 to 40% by mass, and 25 to 35% by mass.

Other materials that can be blended in the hair improving composition according to the present invention are not particularly limited, but examples thereof include oils, which can be any oils used in ordinary cosmetics, regardless of their properties such as natural oils, synthetic oils, or solids, semi-solids, and liquids, and any oils such as hydrocarbons, waxes, fatty acids, higher alcohols, ester oils, silicone oils, and fluorine oils can be used. For example, hydrocarbons such as squalane, squalene, ceresin, paraffin, paraffin wax, liquid paraffin, pristane, polyisobutylene, microcrystalline wax, and petrolatum; waxes such as beeswax, carnauba wax, candelilla wax, and spermaceti wax; animal oils such as beef tallow, beef leg tallow, beef bone tallow, hardened beef tallow, hardened oil, turtle oil, lard, horse oil, mink oil, cod liver oil, and egg yolk oil; lanolin derivatives such as lanolin, liquid lanolin, reduced lanolin, lanolin alcohol, hard lanolin, lanolin acetate, isopropyl lanolin fatty acid, POE lanolin alcohol ether, POE lanolin alcohol acetate, polyethylene glycol lanolin fatty acid, and POE hydrogenated lanolin alcohol ether; fatty acids such as lauric acid, myristic acid, palmitic acid, stearic acid, behenic acid, undecylenic acid, oleic acid, arachidonic acid, docosahexaenoic acid (DHA), isostearic acid, and 12-hydroxystearic acid. Higher alcohols such as lauryl alcohol, myristyl alcohol, palmityl alcohol, stearyl alcohol, behenyl alcohol, hexadecyl alcohol, oleyl alcohol, isostearyl alcohol, hexyldodecanol, octyldodecanol, cetostearyl alcohol, 2-decyltetradecinol, cholesterol, phytosterol, sitosterol, lanosterol, POE cholesterol ether, and monostearyl glycerin ether (batyl alcohol); diisobutyl adipate, 2-hexyldecyl adipate, di-2-heptylundecyl adipate, N-alkyl glycol monoisostearate, isocetyl isostearate, trimethylolpropane triisostearate, ethylene glycol di-2-ethylhexanoate, cetyl 2-ethylhexanoate, trimethylolpropane tri-2-ethylhexanoate, pentaerythritol tetra-2-ethylhexanoate, cetyl octanoate, octyldodecyl gum ester, oleyl oleate, octyldodecyl oleate, decyl oleate, neopentyl glycol dicaprate, triethyl citrate, 2-ethylhexyl succinate, amyl acetate, ethyl acetate, butyl acetate, isocetyl stearate, butyl stearate, diisopropyl sebacate, di-2-ethylhexyl sebacate, cetyl lactate, myristyl lactate, isopropyl palmitate, 2-ethylhexyl palmitate, 2-hexyldecyl palmitate, 2-heptylundecyl palmitate, cholesteryl 12-hydroxystearate, dipentaerythritol fatty acid ester, isopropyl myristate, octyldodecyl myristate, 2-hexyldecyl myristate, myristyl myristate, hexyldecyl dimethyl octanoate, ethyl laurate, hexyl laurate, N-lauroyl-L-glutamic acid-2-octyldodecyl ester, and diisostearyl malate. Further, glyceride oils such as acetoglyceride, glyceryl triisooctanoate, glyceryl triisostearate, glyceryl triisopalmitate, glyceryl tri-2-ethylhexanoate, glyceryl monostearate, glyceryl di-2-heptylundecanoate, and glyceryl trimyristate; silicone oils such as dimethylpolysiloxane, methylphenylpolysiloxane, methylhydrogenpolysiloxane, octamethylcyclotetrasiloxane, decamethylcyclopentasiloxane, dodecamethylcyclohexasiloxane, tetramethyltetrahydrogencyclotetrasiloxane, and higher alkoxy-modified silicones such as stearoxysilicone; silicone-based oils such as higher fatty acid-modified silicones, silicone resins, silicone rubbers, and silicone oils; and fluorine-based oils such as perfluoropolyether, perfluorodecalin, and perfluorooctane.

However, since a sufficient hair improving effect can be obtained with the sulfated polysaccharide salt having the specific structure as described above, which is the hair improver according to the present invention, it is desirable to use a non-silicone composition without blending a silicone-based oil.

Surfactants include anionic, nonionic, and amphoteric surfactants. Examples of anionic surfactants include fatty acid soaps such as sodium stearate and triethanolamine palmitate, alkyl ether carboxylic acids and their salts, carboxylates such as condensates of amino acids and fatty acids, alkyl sulfonates, alkene sulfonates, sulfonates of fatty acid esters, sulfonates of fatty acid amides, sulfonates of alkyl sulfonates and their formalin condensates, alkyl sulfates, higher secondary alcohol sulfates, alkyl and allyl ether sulfates, sulfates of fatty acid esters, sulfates of fatty acid alkylolamides, sulfates such as loto oil, alkyl phosphates, ether phosphates, alkyl allyl ether phosphates, amide phosphates, and N-acylamino acid surfactants.

Examples of cationic surfactants include amine salts such as alkylamine salts, polyamines, and amino alcohol fatty acid derivatives, alkyl quaternary ammonium salts, aromatic quaternary ammonium salts, pyridinium salts, and imidazolium salts. Examples of nonionic surfactants include sorbitan fatty acid esters, glycerin fatty acid esters, polyglycerin fatty acid esters, propylene glycol fatty acid esters, polyethylene glycol fatty acid esters, sucrose fatty acid esters, polyoxyethylene alkyl ethers, polyoxypropylene alkyl ethers, polyoxyethylene alkylphenyl ethers, polyoxyethylene fatty acid esters, polyoxyethylene sorbitan fatty acid esters, polyoxyethylene sorbitol fatty acid esters, polyoxyethylene glycerin fatty acid esters, polyoxyethylene propylene glycol fatty acid esters, polyoxyethylene castor oil, polyoxyethylene hydrogenated castor oil, polyoxyethylene phytostanol ethers, polyoxyethylene phytosterol ethers, polyoxyethylene cholestanol ethers, polyoxyethylene cholesteryl ethers, polyoxyalkylene-modified organopolysiloxanes, polyoxyalkylene-alkyl co-modified organopolysiloxanes, alkanolamides, sugar ethers, and sugar amides. Examples of amphoteric surfactants include betaines, aminocarboxylic acid salts, and imidazoline derivatives.

Examples of metal soaps include aluminum 12-hydroxystearate, zinc stearate, aluminum stearate, calcium stearate, magnesium stearate, zinc myristate, magnesium myristate, zinc cetyl phosphate, calcium cetyl phosphate, sodium zinc cetyl phosphate, zinc laurate, and zinc undecylenate.

Examples of gelling agents include amino acid derivatives such as N-lauroyl-L-glutamic acid and α, γ-di-n-butylamine, dextrin fatty acid esters such as dextrin palmitate, dextrin stearate, and dextrin 2-ethylhexanoate palmitate, sucrose fatty acid esters such as sucrose palmitate and sucrose stearate, benzylidene derivatives of sorbitol such as monobenzylidene sorbitol and dibenzylidene sorbitol, and organically modified clay minerals such as dimethylbenzyldodecylammonium montmorillonite clay and dimethyldioctadecylammonium montmorillonite clay.

Examples of ultraviolet absorbers include cinnamic acid ultraviolet absorbers such as 2-ethylhexyl paramethoxycinnamate, isopropyl paramethoxycinnamate, diethanolamine paramethoxyhydrocinnamate, glyceryl monoparamethoxycinnamate, octyl methoxycinnamate, and methyl diisopropylcinnamate; benzophenone ultraviolet absorbers such as 2-hydroxy-4-methoxybenzophenone, 2-hydroxy-4-methoxybenzophenone-5-sulfonic acid, sodium 2-hydroxy-4-methoxybenzophenone-5-sulfonate, 2,4-dihydroxybenzophenone, 2,2′-dihydroxy-4,4′-dimethoxybenzophenone, 2,2 ′-dihydroxy-4-methoxybenzophenone, 2,2′, 4,4′-tetrahydroxybenzophenone, and 2-hydroxy-4-n-octoxybenzophenone; and benzoic acid ultraviolet absorbers such as paraaminobenzoic acid, ethyl paraaminobenzoate, butyl paraaminobenzoate, 2-ethylhexyl paradimethylaminobenzoate, glyceryl paraaminobenzoate, and amyl paraaminobenzoate.

Examples of ultraviolet protective agents include salicylic acid ultraviolet absorbers such as 2-ethylhexyl salicylate, triethanolamine salicylate, homomenthyl salicylate, dipropylene glycol salicylate, methyl salicylate, ethylene glycol phenyl salicylate, salicylate, amyl salicylate, benzyl salicylate, isopropylbenzyl salicylate, and potassium salicylate; dibenzoylmethane ultraviolet absorbers such as 4-t-butyl-4′-methoxydibenzoylmethane, 4-isopropyldibenzoylmethane, 4-methoxydibenzoylmethane, and 4-t-butyl-4′-hydroxydibenzoylmethane; anthranilic acid ultraviolet absorbers such as menthyl-O-aminobenzoate, 2-phenyl-benzimidazole-5-sulfonic acid, 2-phenyl-5-methylbenzoxazole, 3-(4-methylbenzylidene) camphor, 2-ethylhexyl-2-cyano-3,3-diphenylacrylate, 2-ethyl-2-cyano-3,3′-diphenylacrylate, 2-(2′-hydroxy-5-methylphenyl) benzotriazole, and anthranilic acid ultraviolet absorbers such as menthyl anthranilate; urocanic acid ultraviolet absorbers such as ethyl urocanate; and titanium oxide, zirconium oxide, and cerium oxide.

Examples of alcohols include lower alcohols such as ethanol and isopropanol, in addition to the polyhydric alcohols described above.

Examples of water-soluble polymers include plant-based polymers such as gum arabic, tragacanth, galactan, carob gum, guar gum, karaya gum, carrageenan other than lambda, pectin, agar, alginic acid colloid, fucoidan, torant gum, locust bean gum, and galactomannan; microbial polymers such as xanthan gum, curdlan, gellan gum, fucogel, dextran, succinoglycan, and pullulan; animal polymers such as chitosan, casein, albumin, and gelatin; starch polymers such as starch, carboxymethyl starch, and methylhydroxypropyl starch; cellulose polymers as methylcellulose, such ethylcellulose, methylhydroxypropylcellulose, carboxymethylcellulose, hydroxymethylcellulose, hydroxypropylcellulose, nitrocellulose, sodium cellulose sulfate, sodium carboxymethylcellulose, crystalline cellulose, and cellulose powder; alginic acid polymers such as sodium alginate and propylene glycol alginate; vinyl polymers such as polyvinyl methyl ether, carboxyvinyl polymer, and alkyl-modified carboxyvinyl polymer; polyoxyethylene polymers; polyoxyethylene polyoxypropylene copolymers; acrylic polymers such as sodium polyacrylate, polyethyl acrylate, and polyacrylamide; and inorganic water-soluble polymers such as polyethyleneimine, cationic polymers, bentonite, laponite, and hectorite. This also includes film-forming agents such as polyvinyl alcohol and polyvinylpyrrolidone.

Examples of antibacterial agents include benzoic acid, sodium benzoate, carbolic acid, sorbic acid, potassium sorbate, parahydroxybenzoic acid esters, parachloromethacresol, hexachlorophene, benzalkonium chloride, chlorhexidine chloride, trichlorocarbanilide, photosensitizers, bis(2-pyridylthio-1-oxide)zinc, phenoxyethanol, thiantol, and isopropylmethylphenol. Examples of pH adjusters include potassium carbonate, sodium bicarbonate, and ammonium bicarbonate. Examples of refreshing agents include L-menthol and camphor.

Examples of cell activators include nucleic acid-related substances such as deoxyribonucleic acid and its salts, adenine nucleotide derivatives such as adenosine triphosphate and adenosine monophosphate and their salts, ribonucleic acid and its salts, cyclic AMP, cyclic GMP, flavin adenine dinucleotide, guanine, adenine, cytosine, thymine, xanthine, and their derivatives such as caffeine, theophylline, and their salts; extracts derived from animals such as mammals, birds, shellfish, insects, fish, mollusks, and crustaceans, such as calf blood extract, serum protein-free extract, spleen extract, egg components from birds, comb extract, shell extract, shellfish meat extract, royal jelly, silk protein and its decomposition products or their derivatives, hemoglobin or its decomposition products, lactoferrin or its decomposition products, and squid ink extract; and extracts derived from microorganisms selected from fermentation metabolites such as yeast extract, lactic acid bacteria extract, and bifidobacteria extract. Furthermore, vitamin A such as retinol and its derivatives (retinol palmitate, retinol acetate, etc.), retinal and its derivatives, dehydroretinal, and carotenoids as such carotene; vitamin B such as thiamine (thiamine hydrochloride, thiamine sulfate), riboflavin (riboflavin, riboflavin acetate, etc.), pyridoxine (pyridoxine hydrochloride, pyridoxine dioctanoate, etc.), flavin adenine dinucleotide, cyanocobalamin, folic acid, nicotinic acid (nicotinamide, benzyl nicotinate, etc.), and choline; plant-derived extracts such as apricot extract, ginkgo extract, ginseng extract, barley extract, orange extract, cucumber extract, kiwi extract, shiitake mushroom extract, horsetail extract, swertia japonica extract, jujube extract, capsicum extract, garlic extract, carrot extract, hoelen extract, peach extract, lettuce extract, lemon extract, reishi mushroom extract, rosemary extract, hinokitiol, and cepharanthine; α- and γ-linolenic acid, eicosapentaenoic acid and their derivatives, estradiol and its derivatives and their salts, and organic acids such as glycolic acid, succinic acid, lactic acid, and salicylic acid and their derivatives and their salts. The cell activators listed above can be blended by appropriately selecting one or more types.

Examples of moisturizing agents include mucopolysaccharides such as alkali simple spring water, deep water, hyaluronic acid, chondroitin sulfate, dermatan sulfate, heparan sulfate, heparin, and keratan sulfate, or their salts; proteins such as collagen, elastin, and keratin, or their derivatives and their salts; phospholipids derived from soybean and eggs, glycolipids, ceramides, mucin, honey, erythritol, maltose, maltitol, xylitol, xylose, pentaerythritol, fructose, dextrin and its derivatives, mannitol, sorbitol, inositol, trehalose, and sugars such as glucose; amino acids such as urea, asparagine, aspartic acid, alanine, arginine, isoleucine, ornithine, glutamine, glycine, glutamic acid and its derivatives and their salts, cysteine, cystine, citrulline, threonine, serine, tyrosine, tryptophan, theanine, valine, histidine, hydroxylysine, hydroxyproline, pyrrolidone carboxylic acid and its salts, proline, phenylalanine, methionine, and lysine, and their derivatives or their salts. Furthermore, D-panthenol, avocado extract, almond oil, carob extract, rice extract, strawberry extract, fennel extract, mallow extract, coptis japonica extract, olive oil, lamium album extract, cacao butter, oat extract, ivy extract, bamboo grass extract, gardenia extract, grapefruit extract, geranium thunbergii extract, gentian extract, burdock extract, clematis vitalba extract, sesame extract, cactus extract, saponaria officinalis extract, ginger extract, rehmannia glutinosa extract, shea butter, spiraea extract, cnidium officinale extract, mallow extract, thyme extract, camellia extract, corn extract, cordyceps sinensis extract, tormentil extract, houttuynia cordata extract, ophiopogon japonicus extract, canavalia gladiata extract, witch hazel extract, peppermint extract, spearmint extract, peppermint extract, parsley extract, rose extract, sunflower extract, cypress extract, luffa cylindrica extract, prune extract, butcher's broom extract, borage oil, peony extract, jojoba oil, linden extract, hop extract, pine extract, horse chestnut extract, macadamia nut oil, quince extract, lithospermum erythrorhizon extract, meadowfoam oil, melissa extract, cornflower extract, lily extract, yuzu extract, lime extract, lavender extract, gentian extract, sanguisorba officinalis extract, and apple extract. The moisturizing agents listed above can be blended by appropriately selecting one or more types.

Examples of vitamins include vitamin K such as phytonadione, menaquinone, menadione, and menadiol, vitamin P such as eriocitrin and hesperidin, biotin, carnitine, and ferulic acid. Examples of blood circulation promoters include nonanoic acid vanillylamide, capsaicin, zingerone, cantharidin tincture, ichthammol, α-borneol, inositol hexanicotinate, cyclandelate, cinnarizine, trazoline, acetylcholine, verapamil, and γ-oryzanol. Examples of skin astringents include tannic acid, examples of antiseborrheic agents include thiantol, and examples of enzymes include lipase and papain. One or more of these components can be appropriately added. The amount of these components added is not particularly limited, but it can usually be added in the range of 0.0001 to 80% by mass (based on the total amount).

The hair improving composition of the present invention can be produced according to a usual method, and can be applied as, for example, shampoo, hair rinse, hair conditioner, hair treatment, hair cream, styling lotion, styling mousse, conditioning mousse, hair spray, hair mist, hair foam, hair gel, and hair blow.

The hair improver or hair improving composition according to the present invention is effective not only for humans, but also for mammals including livestock such as cattle, horses, pigs, and goats, and pets such as dogs and cats.

### EXAMPLES

Next, the present invention will be described in more detail with reference to examples, but the present invention is not limited to these examples. In the following examples, the unit % of the numerical value indicating the addition amount of each component means % by mass.

The effects of the hair improver according to the present invention and the comparative drug on hair were evaluated for hair moisture content, combability, and shine using damaged hair prepared by the following method.

### EXAMPLE 1

### Preparation of Damaged Hair

A hair bundle obtained from a collaborator was immersed in a bleaching agent for 30 minutes, and then the bleaching agent was washed away with running water. This treatment was performed three times to prepare damaged hair. For the test, the hair bundle left to stand in a constant temperature and humidity room (room temperature 21° C., humidity 45%) for 24 hours was used as damaged hair.

### Hair Moisture Content

The damaged hair prepared above was immersed in tap water at 30° C. for 1 minute, and then left to stand in a constant temperature and humidity room for 18 hours, and the hair weight (mg) was measured (before application). For the test product, 500 μL of the sample was applied to the damaged hair bundle, immersed in tap water at 30° C. for 1 minute, left to stand in a constant temperature and humidity room for 18 hours, and then the hair weight (mg) was measured (after application). The difference in hair weight before and after application was defined as hair moisture content, and compared.

### Hair Combability

The damaged hair prepared above was immersed in tap water at 30° C. for 1 minute, and then left to stand in a constant temperature and humidity room for 18 hours. A comb was attached to a digital force gauge, and the load (N) applied to the comb when combing the hair bundle was measured as an index of the frictional force generated between the hair and the comb (before application). For the test product, 500 μL of the sample was applied to the damaged hair bundle, immersed in tap water at 30° C. for 1 minute, left to stand in a constant temperature and humidity room for 18 hours, and then the hair friction force (N) was measured (after application). Based on the hair friction force before and after application, the effect of the sample on improving combability was evaluated. In the results, the smaller the hair friction force, the better the combability.

### Hair Shine

The damaged hair prepared above was immersed in tap water at 30° C. for 1 minute, and then left to stand in a constant temperature and humidity room for 18 hours, and an image was taken using a digital single-lens reflex camera (before application). For the test product, 500 μL of the sample was applied to the damaged hair bundle, immersed in tap water at 30° C. for 1 minute, left to stand in a constant temperature and humidity room for 18 hours, and then an image of the damaged hair was taken (after application).

The luminance (a.u.) of the captured hair image was quantified by image analysis software, and the full width at half maximum was calculated from the measured distance (pixels) of the quantified hair luminance (a.u.), and used as the hair shine index. Image analysis was performed based on the hair images before and after application, and the width of the bright hair part (so-called “angel's halo”), which is the hair shine index, was evaluated to evaluate the effect on hair shine. The narrower the full width at half maximum, the less irregular reflection of light, indicating that the hair has shine.

### Sample Preparation

Water was added to the powdered dried seaweed funori, and the mixture was dissolved so as to loosen the entangled structure of the components. The dissolved part and the undissolved part were accurately separated into molecular units. Sodium chloride was added to the solution, and the mixture was heated. After heating, it was purified by separating by molecular weight using a molecular weight sieve, and the sodium salt of the 6-position O-sulfated polysaccharide (hereinafter referred to as “funoran Na”) was taken out.

The constituent sugars of this product were analyzed by the hydrolysis method and TLC analysis method (1-naphthol sulfuric acid color development method), which will be described in detail below, and the constituent sugar was shown to be galactose (galactose) Gal. Furthermore, as a result of analysis by the structural analysis method using ¹³C-NMR, which will be described in detail below, it was found that it is a Gal 6-position sulfated polysaccharide, and that Gal without a sulfate group has a structure in which it is bonded via the 3-position and 6-position oxygen (O).

### <Analysis of Constituent Sugars>

### Test Method

To 1 g of funoran Na, 25 mL of purified water and 1 mL of concentrated sulfuric acid were added, and the mixture was heated under reflux. After hydrolysis, the mixture was neutralized with 2 g of sodium carbonate, 1 mL of the hydrolysis solution was taken, 9 mL of methanol was added, and the mixture was centrifuged. The supernatant of the centrifugation was used as the test solution.

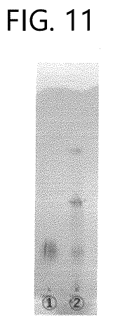

In thin layer chromatography (TLC), a solution prepared by adding 1 mL of water and 9 mL of methanol to 10 mg of D (+)−galactose was used as a standard solution. After spotting 10 pL each of the test solution and the standard solution on 5 cm ×20 cm TLC, the mixture was developed with a developing solvent (acetone:water=9:1) and air-dried. This TLC was sprayed with 1-naphthol sulfuric acid reagent (Japanese Pharmacopoeia) and colored at 105° C. After developing the TLC, it was dried and colored. As shown in FIG. 11, a reddish-brown spot was observed at the same Rf value as the galactose standard solution: 0.20. This showed that funoran Na has galactose as a constituent sugar.

### <Structural Analysis by ¹³C-NMR>

### Test Method

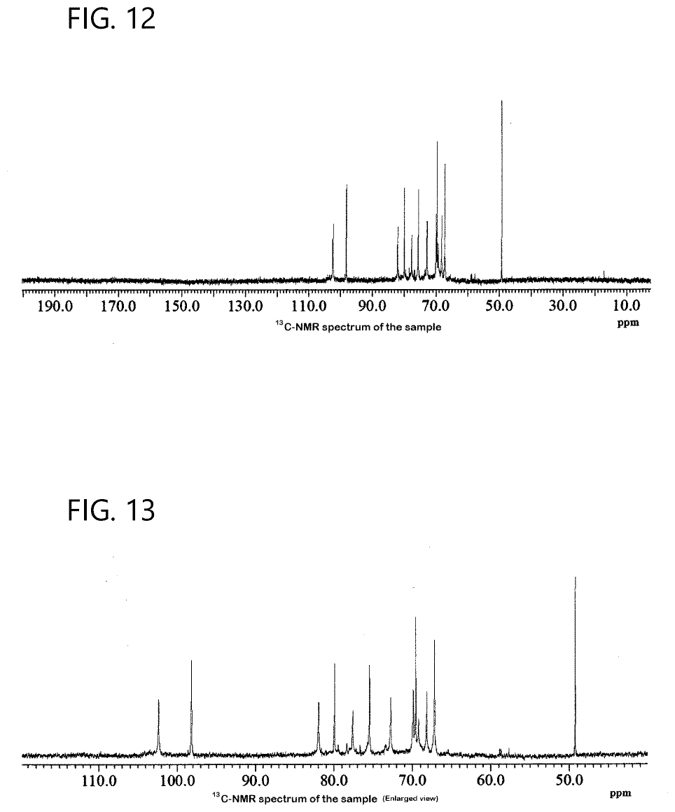

To 80.4 mg of funoran Na, 1.5 mL of heavy water and 0.005 mL of methanol were added and dissolved at 80° C. ¹³C-NMR of the obtained sample deuterium solution was measured using a nuclear magnetic resonance analyzer (JNM-ECZ600R, JEOL Ltd.) at a magnetic field strength of 14.1 T (1H resonance frequency: 600 MHZ). The obtained results are shown in FIGS. 12 and 13. As shown in FIGS. 12 and 13, the chemical shift of the C6 carbon of β-D-Gal-6-SO³⁻ was observed near 67 ppm, and the chemical shift of the carbon of the sulfate sugar was observed at C1:102 ppm, C2:70 ppm, C3:82 ppm, C4:68 ppm, C5:73 ppm.

The chemical shift of the carbon of 3,6-anhydro-α-L-Gal is observed at C1:98 ppm, C2:70 ppm, C3:80 ppm, C4:77.5 ppm, C5:75.5 ppm, C6:69 ppm, so it can be seen that the structure is as follows.

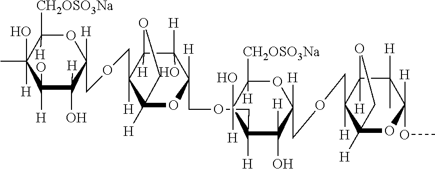

### Experiment

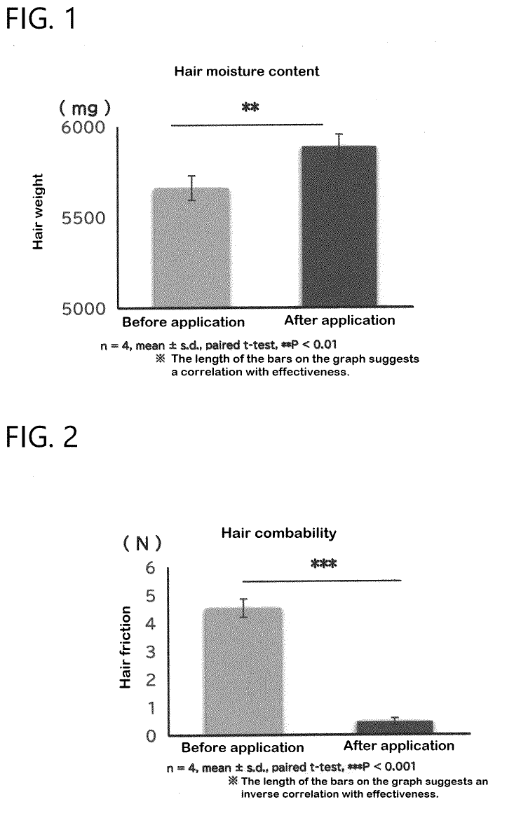

An aqueous solution containing 0.001% of the obtained 6-position O-sulfated polysaccharide sodium salt and 30% of naturally derived 1,3-butylene glycol was prepared and used as a sample (sample 1), and subjected to the experiments related to hair moisture content, hair combability, and hair shine described above. The obtained results are shown in FIGS. 1, 2, and 3.

As shown in FIG. 1, sample 1 according to the example of the present invention significantly increased hair weight compared to before application. From this, it can be expected that the hair improver according to the present invention has the effect of increasing hair moisture content and improving hair moisturizing power.

As shown in FIG. 2, sample 1 according to the example of the present invention significantly reduced the hair friction force index compared to before application. From this, it was observed that the hair improver according to the present invention improves combability, and it was considered that it has the effect of giving smoothness to hair.

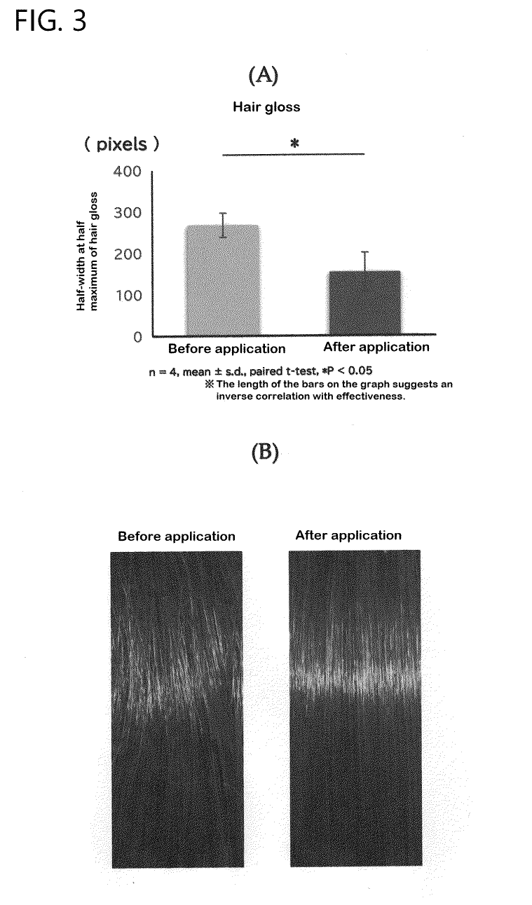

Furthermore, as shown in FIG. 3, sample 1 according to the example of the present invention significantly improved the hair shine index compared to before application. From this, it was considered that the hair improver according to the present invention suppresses hair frizz and gives visual shine.

### EXAMPLE 2

In order to further investigate whether the sample according to the example of the present invention has an effect like a finishing agent that has an effect on hair as it is without applying it to damaged hair, the evaluation method of Example 1 was slightly modified and carried out. That is, as shown below, each evaluation was performed without performing the water immersion treatment after applying the sample to damaged hair.

### Preparation of Damaged Hair

Damaged hair was prepared in the same manner as shown in Example 1.

### Hair Moisture Content

The damaged hair prepared above was immersed in tap water at 30° C. for 1 minute, and then left to stand in a constant temperature and humidity room for 18 hours, and the hair weight (mg) was measured (before application). For the test product, 500 μL of the sample was applied to the damaged hair bundle, and then left to stand in a constant temperature and humidity room for 18 hours, and the hair weight (mg) was measured (after application). The difference in hair weight before and after application was defined as hair moisture content, and compared.

### Hair Combability

The damaged hair prepared above was immersed in tap water at 30° C. for 1 minute, and then left to stand in a constant temperature and humidity room for 18 hours. A comb was attached to a digital force gauge, and the load (N) applied to the comb when combing the hair bundle was measured as an index of the frictional force generated between the hair and the comb (before application). For the test product, 500 μL of the sample was applied to the damaged hair bundle, and then left to stand in a constant temperature and humidity room for 18 hours, and the hair friction force (N) was measured (after application). Based on the hair friction force before and after application, the effect of the sample on improving combability was evaluated. In the results, the smaller the hair friction force, the better the combability.

### Hair Shine

The damaged hair prepared above was immersed in tap water at 30° C. for 1 minute, and then left to stand in a constant temperature and humidity room for 18 hours, and an image was taken using a digital single-lens reflex camera (before application). For the test product, 500 μL of the sample was applied to the damaged hair bundle, and then left to stand in a constant temperature and humidity room for 18 hours, and an image of the damaged hair was taken (after application).

The luminance (a.u.) of the captured hair image was quantified by image analysis software, and the full width at half maximum was calculated from the measured distance (pixels) of the quantified hair luminance (a.u.), and used as the hair shine index. Image analysis was performed based on the hair images before and after application, and the width of the bright hair part (so-called “angel's halo”), which is the hair shine index, was evaluated to evaluate the effect on hair shine. The narrower the full width at half maximum, the less irregular reflection of light, indicating that the hair has shine.

### Sample Preparation

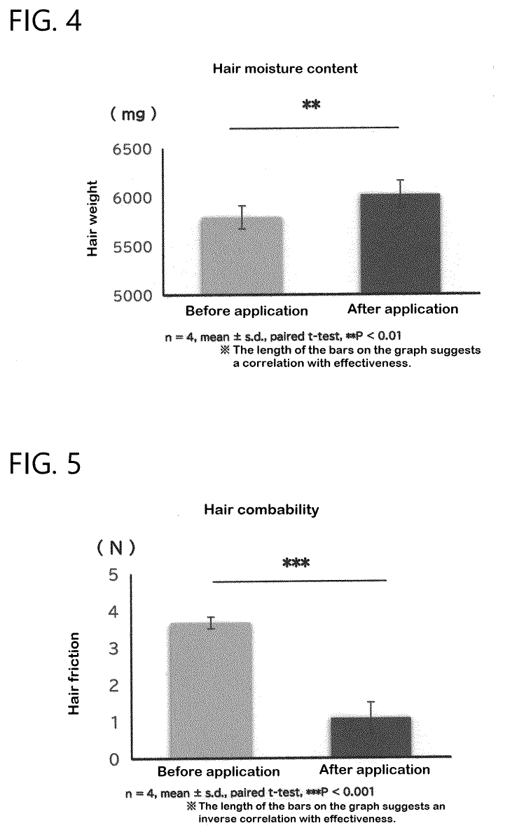

The 6-position O-sulfated polysaccharide sodium salt was produced from dried seaweed funori in the same manner as in Example 1. Then, an aqueous solution containing 0.0001% of this 6-position O-sulfated polysaccharide sodium salt and 30% of naturally derived 1,3-butylene glycol was prepared and used as a sample (sample 2), and subjected to the experiments related to hair moisture content, hair combability, and hair shine described above. The obtained results are shown in FIGS. 4, 5, and 6.

As shown in FIG. 4, sample 2 according to the example of the present invention significantly increased hair weight compared to before application. From this, it can be expected that the hair improver according to the present invention has the effect of increasing hair moisture content and improving hair moisturizing power.

As shown in FIG. 5, sample 2 according to the example of the present invention significantly the hair friction force index compared to before application. From this, it was observed that the hair improver according to the present invention improves combability, and it was considered that it has the effect of giving smoothness to hair.

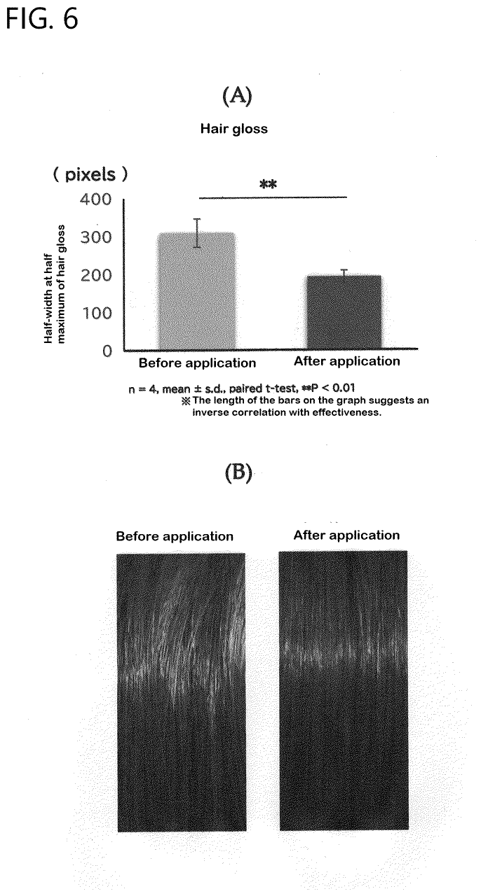

Furthermore, as shown in FIG. 6, sample 1 according to the example of the present invention significantly improved the hair shine index compared to before application. From this, it was considered that the hair improver according to the present invention suppresses hair frizz and gives visual shine.

### EXAMPLE 3

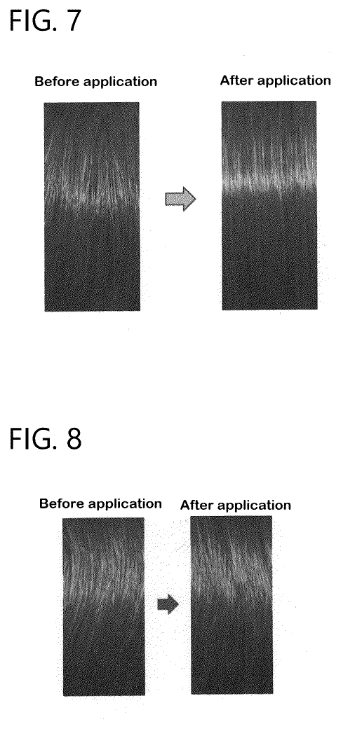

Except that the 6-position O-sulfated polysaccharide sodium salt obtained by the synthesis method was used as the sample for the experiment, an aqueous solution containing 0.001% of the 6-position O-sulfated polysaccharide sodium salt and 30% of naturally derived 1,3-butylene glycol was prepared in the same manner as in Example 1 and used as a sample (sample 3), and this sample was subjected to the experiment related to hair shine in the same manner as in Example 1. The obtained results are shown in FIG. 7.

As shown in FIG. 7, sample 3 according to the example of the present invention improved hair shine compared to before application. From this, it was considered that the hair improver according to the present invention suppresses hair frizz and gives visual shine even when it is obtained by the synthesis method.

### COMPARATIVE EXAMPLE 1

Using the sulfated polysaccharide sodium salt having a sulfate group at the 2- and 4-positions but not at the 6-position obtained by the synthesis method as the sample for the experiment, an aqueous solution containing 0.001% of this sulfated polysaccharide sodium salt and 30% of naturally derived 1,3-butylene glycol was prepared and used as a sample (comparative sample 1), and this sample was subjected to the experiment related to hair shine in the same manner as in Example 1. The obtained results are shown in FIG. 8.

As shown in FIG. 8, comparative sample 1 did not visually improve hair shine compared to before application.

### COMPARATIVE EXAMPLE 2

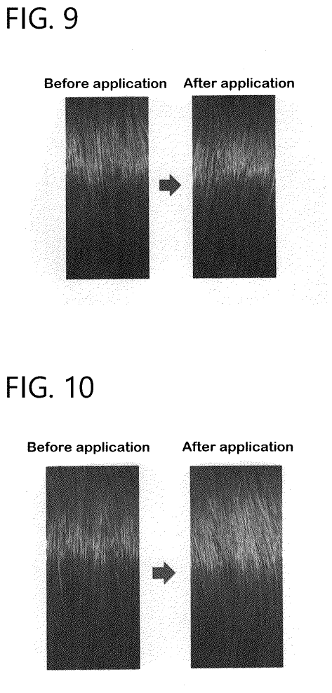

Except that calcium chloride was used instead of sodium chloride in Example 1, the 6-position O-sulfated polysaccharide calcium salt was produced in the same manner as in Example 1. Then, an aqueous solution containing 0.001% of the obtained 6-position O-sulfated polysaccharide calcium salt and 30% of naturally derived 1,3-butylene glycol was prepared and used as a sample (comparative sample 2), and this sample was subjected to the experiment related to hair shine in the same manner as in Example 1. The obtained results are shown in FIG. 9.

As shown in FIG. 9, comparative sample 2 did not visually improve hair shine compared to before application.

### COMPARATIVE EXAMPLE 3

As a sample for the experiment, 100 ml of water was added to 1 g of the same powdered dried seaweed funori as used in Example 1, and the mixture was thoroughly mixed to prepare a funori solution, and then 1 ml of this funori solution was diluted with 100 ml of water, and this was used as a sample (comparative sample 3), and this sample was subjected to the experiment related to hair shine in the same manner as in Example 1. The obtained results are shown in FIG. 10.

As shown in FIG. 10, comparative sample 3, which is a common funori blend, had a higher concentration of funori components than Example 1, but did not visually improve hair shine compared to before application.

What is claimed is:

1. A hair improver comprising a sulfated polysaccharide salt having a sulfate group at the 6-position of at least one sugar unit structure and salted with a monovalent cation.

2. The hair improver according to claim 1, wherein the sugar constituent units of the polysaccharide have one or more hexacarbohydrate constituent units selected from the group consisting of fucose, rhamnose, arabinose, galactose, glucose, xylose, mannose, fructose, and ribose, and at least one of the hexacarbohydrate unit structures has a sulfate group at the 6-position.

3. The hair improver according to claim 1, wherein the sulfated polysaccharide salt is represented by any of the following compound structural formulae: 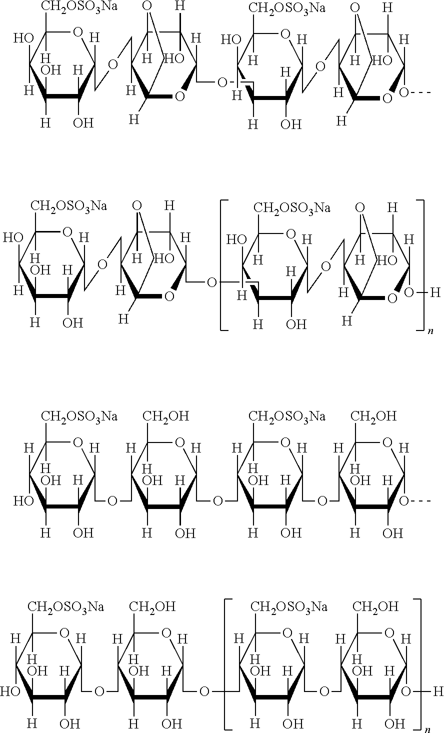
(Provided that, in these formulae, n represents a number from 1 to 20000).

4. A hair improving composition comprising the hair improver according to claim 1 in an amount of 0.000005% by mass to 0.01% by mass.

5. The hair improving composition according to claim 5, which is an aqueous composition further containing a polyhydric alcohol in an amount of 10 to 50% by mass.
